(19) United States
(12) Patent Application Publication
(10) Pub. No.: US 2010/0100111 A1
(43) Pub. Date: Apr. 22, 2010

(54) APPARATUS AND METHOD FOR SURGERY

(76) Inventor: John S. Rogerson, Madison, WI (US)

Correspondence Address: COOK ALEX LTD, SUITE 2850, 200 WEST ADAMS STREET, CHICAGO, IL, 60606, US

(21) Appl. No.: 12/255,147

(22) Filed: Oct. 21, 2008

(51) Int. Cl. A61B 17/32

(52) U.S. Cl. 606/167; 606/190

(57) ABSTRACT

The disclosure pertains to a surgical implement that has a gripping end and a surgical end. The surgical end includes a first side, a second side opposite the first side and a pointed distal end wherein the first side and the distal end are blunt and the second side has a surgically sham cutting edge. This surgical implement can be utilized during surgical procedures to maintain an incision in an open position or enlarge or modify an incision to accommodate an endoscope and other surgical implements. The use of this surgical implement to cut, expand and maintain an open incision saves time and minimizes trauma to the tissue.

### TECHNICAL FIELD

The present approach generally relates to surgical implements and methods for use in surgery and in particular arthroscopic surgery.

### BACKGROUND

It is generally known that surgeons attempt to limit the size of a surgical incision to a size that is just sufficient for the surgical procedure to be performed so that trauma to the tissue is minimized. Arthroscopy, for example, is a minimally invasive method used in orthopedic surgery to evaluate and treat joint injuries. In arthroscopy surgical procedures, an arthroscopic portal is made with one or more small incisions, usually less than one inch in width, at the site of treatment. An endoscope may be inserted into the portal to view the injured joint. In addition, various surgical implements may be inserted into the portal to effect treatment. During these procedures it may be necessary to maintain the incisions in an open position by employing, for example, a switching stick or enlarge or modify the incisions by utilizing, for instance, a scalpel, to accommodate the endoscope and other surgical implements.

Goals that have been arrived at in accordance with the present approach, while maintaining the benefits associated with minimal incisions, include surgical instruments that more efficiently open, enlarge or modify incisions and more efficient surgical procedures to open, enlarge or modify incisions. Other goals include surgical instruments and methods suited for a sterile surgical environment and saving time in the cutting, expanding and maintaining an incision.

Prior art approaches that have not recognized the positives that could be gained by seeking to achieve these types of objectives or teach solutions other than those of the present approach include U.S. patent publication number 2002/0143356 that describes a craniotome. The device, however, includes a guard that prevents the cutting portion of the craniotome from contacting the dura during the formation of a bone flap to access the brain. U.S. patent publication number 2004/0267269 pertains to a cutting device to form a cavity in bone, either to implant a prosthesis into the cavity or to harvest a bone sample. The device includes a hollow handle portion through which passes a flexible shaft at the end of which is carried a cutting member. Once the cutting portion is in place, the flexible shaft can be manipulated so that the cutting member creates a void in the bone. U.S. patent publication number 2005/0251191 describes a blunt tip obturator—essentially a type of switching stick that is in common use now. The cross-section of the obturator is described as having a shape, which both decreases tenting of the tissue being passed through, and decreases the amount of force required to pass the obturator through the initial incision. The device does not include a blade of any description. U.S. patent publication number 2006/0111722 relates to a scalpel-like instrument having a golf-club appearance, with a blade disposed on the “toe” portion of the club.

U.S. patent publication number 2006/0149267 relates to a safety knife for widening an annular opening. The device includes a handle portion and a working end having a blade disposed between two blunt and gently rounded “teeth.” The blade, however, can only cut tissue that can be manipulated to fit between the two blunt teeth of the device. U.S. patent publication number 2006/0229656 pertains to a bona fide switching stick. The apparatus is a blunt switching stick paired with two shoehorns to widen an initial incision. U.S. patent publication number 2007/0010842 describes a trocar having an asymmetrical tip that bears a cutting surface. The cutting surface can be exposed to make the initial incision. A sliding shield can then be moved to cover the cutting surface and the device used in standard fashion as a trocar or stitching stick. U.S. Pat. No. 5,066,288 is directed to a safety trocar. A cutting surface is exposed to make an initial incision. A sliding shield can then be moved to cover the cutting surface and the device used in standard fashion as a trocar or stitching stick. The distal end where the blade is disposed is hemispherical in shape, with the blade extending from the hemisphere to make the incision. U.S. Pat. No. 5,116,351 describes what is otherwise a standard scalpel having a retractable hood to protect the blade. The retractable hood is spring-loaded. U.S. Pat. No. 5,582,618 describes a two-bladed cutting device having cutting surfaces on opposing arms of a pair of reciprocating jaws. When the jaws are closed, the cutting members cut the tissue within the jaws. A blade disposed within it and protected by a protruding portion. U.S. Pat. No. 5,674,237 relates to a safety trocar. Here, the cutting tip is a pyramidal cross-section and three spring-loaded shields extend up each face of the three-sided pyramid to shield the cutting surfaces when only an obturator tip is needed. U.S. Pat. No. 5,833,692 pertains to a surgical instrument in which a cutting blade can be carried to the surgical location by passing it through a long, hollow shaft. The shaft is hollow at a point to allow the distal, working end of the device to be manipulated within the surgical site. U.S. Pat. No. 6,270,501 discloses a cannula having a two-bladed scalpel incorporated within the shaft of the cannula. The shaft of the cannula is configured to fit onto the end of a guide pin. The cannula, with the cutting head attached, is then guided through the tissue/bone to be cut.

With the present approach, it has been determined that various characteristics of prior art, such as these patents, have shortcomings and undesirable attributes, results or effects. The present approach recognizes and addresses matters such as these to provide enhancements not heretofore available. Overall, the present approach provides more fully enhanced surgical instruments and procedures that open, modify and enlarge incisions.

### SUMMARY

In an embodiment of the present approach a surgical implement comprises a gripping end and a surgical end that includes a first side, a second side opposite the first side and a distal pointed end wherein the first side and the distal pointed end are blunt and the second side has a surgically sharp cutting edge. This surgical implement can be utilized during surgical procedures to maintain an incision in an open position or enlarge or modify an incision to accommodate an endoscope and other surgical implements. The use of this surgical implement to cut, expand and maintain an open incision saves time and minimizes trauma to the tissue. Furthermore, the surgical implement of the present approach permits a user to expand an incision in a tough tissue, such as the joint capsule, without having to remove all surgical implements already present.

In another embodiment of the present approach, the second side of the surgical end is blunt around the cutting edge such that the cutting edge is depressed so that unwanted or accidental cutting is prevented.

In an additional embodiment of the present approach the surgical cutting edge is integrally formed in the surgical instrument.

In a further embodiment of the present approach a separate blade with a surgical cutting edge is embedded in the surgical instrument.

In still another embodiment of the present approach a disposable blade with a surgical cutting edge is releasably locked in the surgical instrument.

### BRIEF DESCRIPTION OF THE DRAWINGS

FIG. 1 is a side plan view of an embodiment of a surgical implement according to the present approach.

FIG. 2 is a partial plan view of the bottom of the surgical implement shown in FIG. 1.

FIG. 3 is a partial plan view of the side of the surgical implement shown in FIG. 1.

FIG. 4 is a partial plan view of the top of the surgical implement shown in FIG. 1.

FIG. 5 is a plan view of a side of a disposable blade with a surgical cutting edge according to the present approach.

FIG. 6 is a partial side view of a disposable blade as shown in FIG. 5 partially inserted into a surgical implement.

FIG. 7 is a cross section view taken along lines 7-7′ as shown in FIG. 6.

FIG. 8 is a partial side view of a disposable blade as shown in FIG. 5 fully inserted into a surgical implement.

FIG. 9 is a cross-sectional view of a schematic of a body joint.

### DETAILED DESCRIPTION

As required, detailed embodiments of the present approach are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner, including employing various features disclosed herein in combinations that might not be explicitly disclosed herein.

The present approach is directed to a surgical implement. In an embodiment, the implement can be used in arthroscopic surgical procedures for the examination and treatment of joints, for example, the implement can be used to open, expand or modify incisions made during arthroscopic surgery. FIG. 1 represents an embodiment according to the present approach wherein a surgical implement, generally shown as 10, comprising a handle or gripping end 12 and a surgical functional end, generally shown as 14. Length, L1, of implement 10 can be, for example, between about 12 cm and about 22 cm, typically between about 15 cm and about 19 cm. Gripping end 12 can be approximately cylindrical with a diameter, D1, for instance, between about 2 mm to about 6 mm, typically about 3 mm to about 5 mm. Gripping end 12 can be used as a handle for manipulating implement 10 and can also be used to maintain an incision in an open position. The manipulation can be digital or implement 10 can be mechanically supported and manipulated with a mechanical device, for example, employing electrosurgical devices for laparoscopic surgery.

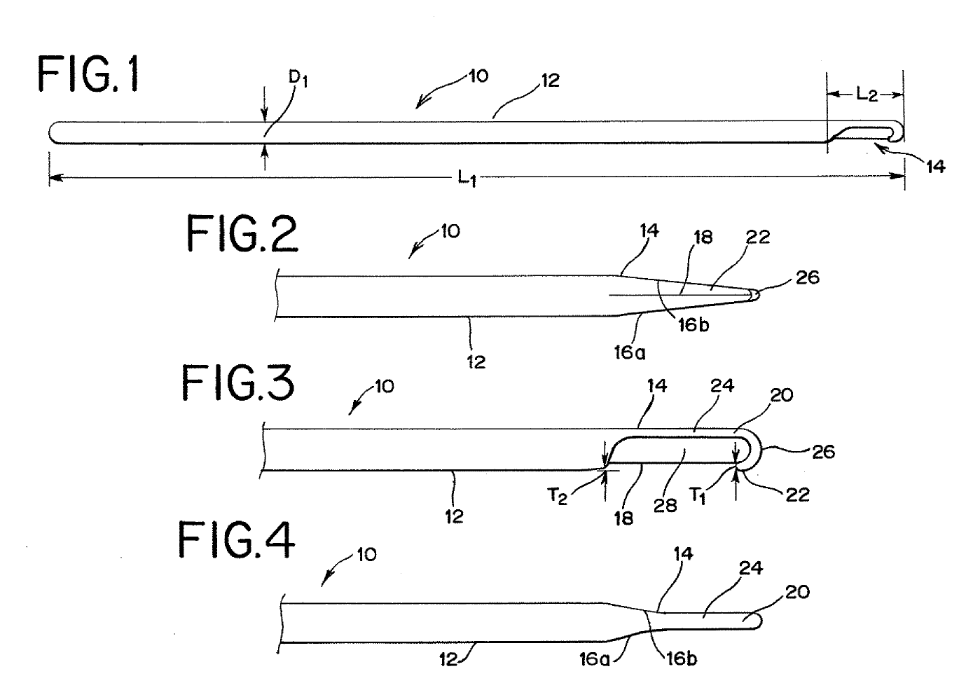

As depicted in FIGS. 2-4, surgical end 14 begins with implement 10 tapering along two planes 16a and 16b. Length, L2, of surgical end 14 can be, for example, between about 10 mm and 20 mm, suitably between about 13 mm and about 17 mm. In this embodiment, surgical end 14 also includes a surgically sharp cutting edge 18 and a blunt portion 20. Cutting edge 18 is present only at first side 22 considered a lower side of surgical end 14. Blunt portion 20 is present on a second side 24, considered an upper side and opposite lower side 22 and on a distal pointed end 26 of implement 10. As shown in FIG. 3, blunt portion 20 can also extend to lower side 22. That is, blunt portion 20 can be present on upper side 24, distal end 26 and lower side 22 except for the length of cutting edge 18 present on lower side 22. In one embodiment, the blunt portion 20 on lower side 22 has a thickness, T1, and cutting edge 18 is set back a distance, T2, from the outside surface of gripping end 12. Both T1 and T2 can be for instance, between about 0.50 mm and about 1.50 mm, typically, between about 0.75 mm and about 1.25 mm. Cutting edge 18 is thus depressed within surgical end 14 such that tissue, cartilage and bone are protected from accidental or unwanted cutting by cutting edge 18.

Implement 10 can be made of suitable surgical materials such as surgical grade stainless steel and like metals or biocompatible plastic materials that are easily sterilized. When surgical metals are employed cutting edge 18 can be integrally part of the metal by molding, grinding and sharpening cutting edge 18 from the metal utilized for implement 10. Alternatively, cutting edge 18 can be formed from a scalpel blade 28 or similar devices effective for cutting tissue using surgical metals or biocompatible plastic materials and placed, inserted or embedded in surgical end 14. Implement 10 can be re-useable either completely or in part and implement 10 may be composed of materials that are suited to be repeatedly washed and sterilized.

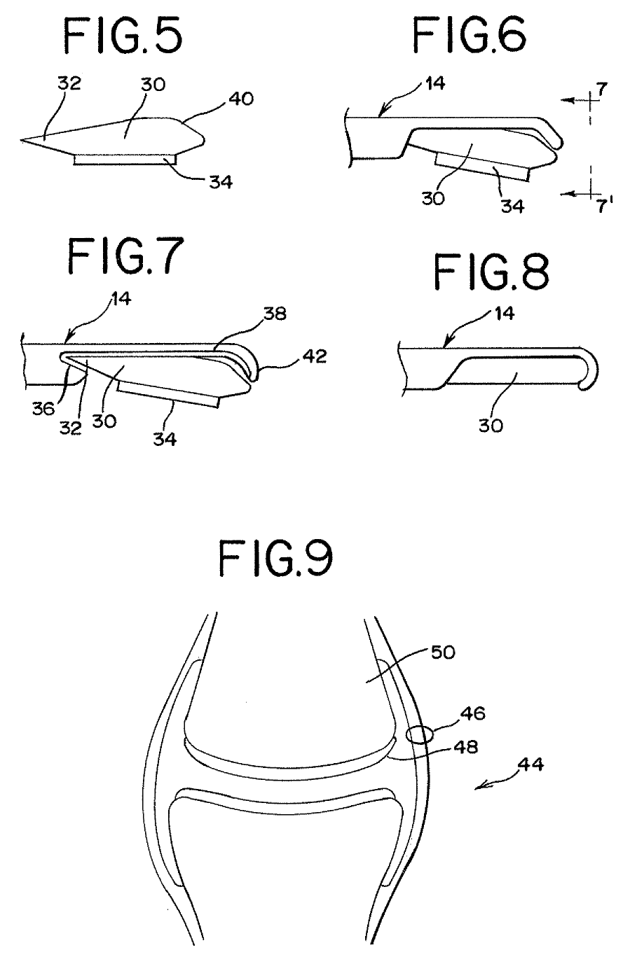

In an embodiment as illustrated in FIGS. 5-8, a disposable, single scalpel blade 30 of a desired size is releasably snapped locked in surgical end 14. Disposable scalpel blade 30 has an insertion tip 32 and a protective cap 34, suitably plastic. To insert disposable scalpel blade 30 into surgical end 14, insertion tip 32 is first inserted into cavity 36 of surgical end 14. As disposable scalpel blade 30 is urged towards a blade holding groove 38 of surgical end 14, camming surface 40 of disposable scalpel blade 30 engages holding tip 42 of blunt portion 20 urging blunt portion 20 in a direction considered outwardly. Disposable scalpel blade 30 is thus permitted to set in blade holding groove 38. After camming surface 40 passes holding tip 42, holding tip 42 returns to its original position, thereby releasably snap locking disposable scalpel blade 30 in surgical end 14. Protective cap 34 is then removed. Disposable scalpel blade 30 can be removed by pulling with a gripping device such as pincers so that a new scalpel blade can be inserted.

In another embodiment, the entire surgical end 14 of implement 10 can be detachable from gripping end 12 so that a different combination of gripping end 12 and surgical end 14 can be formed. In a further embodiment, implement 10 can be used in conjunction with other surgical implements. For example, implement 10 can be passed through the lumen of a cannula to a treatment site.

The surgical implement according to the present approach can be used to maintain, modify or enlarge incisions made, for example, during arthroscopic surgery. According to one method of use, implement 10 can be inserted into an incision where the first gripping end 12 may be used as handle for the user to help maintain an incision in an open position. Cutting edge 18 can be used to cut tissue to enlarge or modify the incision. Blunt portion 20 can act as a switching stick and bring pressure to the walls of an incision to open up or maintain the incision as required.

The surgical implement of the present approach can be used to be make highly selective incisions during surgery such that particular structures may be cut while adjacent structures are not cut. For example, the surgical implement 10 can be manipulated within an incision so that the cutting edge 18 may be used to cut tissue while blunt portion 20 can be used to ensure that adjacent tissues are not exposed to the cutting edge.

In an embodiment, implement 10 can be used during treatment of joint 44 as shown in FIG. 9. Implement 10 can be manipulated to cut strong capsule tissue 46 of joint 44 while not affecting articular cartilage 48 or bone 50. The use of a single surgical instrument to cut, expand and maintain an incision saves time and minimizes trauma to the tissue. For example, the surgical implement of the present approach permits a user to expand an incision in a tough tissue, such as the joint capsule, without having to remove all surgical implements already present. Thus, implement 10 saves time during surgery and thereby reducing the risk of trauma and accidents during surgery.

It will be understood that the embodiments of the present disclosure are illustrative of some of the applications of the principles associated with the present approach. Numerous modifications may be made by those skilled in the art without departing from the true spirit and scope of the disclosure. Various features, which are described herein, can be used in any combination and are not limited to particular combinations that are specifically described herein.

1. A surgical implement, comprising:
a) a gripping end; and
b) a surgical end including a first side, a second side opposite the first side and a pointed distal end wherein the first side and the pointed distal end are blunt and the second side has a surgically sharp cutting edge.

2. The surgical implement according to claim 1 further comprising at least one blunt edge on the second side next to the cutting edge.

3. The surgical implement according to claim 2 wherein the cutting edge has a blunt edge on each side of the cutting edge and the cutting edge is depressed by the thickness of each blunt edge.

4. The surgical implement according to claim 3 wherein the thickness of the blunt surrounding edge is between about 0.50 mm and about 1.50 mm.

5. The surgical implement according to claim 4 wherein the thickness of the blunt surrounding edge is between about 0.75 mm and about 1.25 mm.

6. The surgical implement according to claim 1 wherein the surgical implement is comprised of materials selected from the group consisting or surgical grade metals and surgical grade biocompatible plastics.

7. The surgical implement according to claim 6 wherein the surgical implement is comprised of surgical grade metals.

8. The surgical implement according to claim 7 wherein the surgical implement is comprised of surgical grade stainless steel.

9. The surgical implement according to claim 7 wherein the cutting edge is integral with the surgical implement.

10. The surgical implement according to claim 7 further comprising a blade comprising the cutting edge wherein the blade is embedded in the surgical instrument.

11. The surgical implement according to claim 1 further comprising a blade holding channel in the surgical end and a separate blade with the cutting edge wherein the blade is releasably locked in the blade holding channel.

12. The surgical implement according to claim 11 further comprising a blade cavity in the surgical end and the blade further comprising an insertion tip and a camming edge engagable with a blunt end on the second side.

13. The surgical implement according to claim 1 wherein the length of the surgical instrument is between about 12 cm and about 22 cm.

14. The surgical implement according to claim 13 wherein the length of the surgical instrument is between about 15 cm and about 19 cm.

15. The surgical implement according to claim 1 wherein the length of the surgical end is between about 10 mm and about 20 mm.

16. The surgical implement according to claim 15 wherein the length of the surgical end is between about 13 mm and about 22 mm.

17. The surgical implement according to claim 1 wherein the diameter of the gripping end is between about 2 mm and about 6 mm.

18. The surgical implement according to claim 18 wherein the diameter of the gripping end is between about 10 mm and about 20 mm.

19. A surgical method comprising:
a) making a surgical incision;
b) selecting a surgical implement having;
i) a gripping end; and
ii) a surgical end including a first side, a second side opposite the first side and a pointed distal end wherein the first side and the pointed distal end are blunt and the second side has a surgically sharp cutting edge;
c) opening the incision with the selected surgical implement; and
d) cutting tissue with the selected surgical implement.

20. The surgical method according to claim 20 wherein the surgical method is arthroscopic surgery.
